(19) United States
(12) Patent Application Publication
(10) Pub. No.: US 2020/0350068 A1
(43) Pub. Date: Nov. 5, 2020

(54) Portable Medical Data Hub

(71) Applicant: SANOFI, Paris (FR)

(72) Inventor: Michael Helmer, Frankfurt am Main (DE)

(21) Appl. No.: 16/754,562

(22) PCT Filed: Oct. 9, 2018
(86) PCT No.: PCT/EP2018/077429

§ 371 Date: Apr. 8, 2020

(30) Foreign Application Priority Data
Oct. 10, 2017 (EP) 17306363.7

(51) Int. Cl. G16H 40/67; G16H 50/20; G16H 10/60; G16H 40/40; G16H 20/10; G06K 7/10; G06K 7/14

(52) U.S. Cl. CPC G16H 40/67; G16H 50/20; G16H 10/60; G16H 40/40; G06F 3/0488; G06K 7/10366; G06K 7/1413; G06K 7/1417; G16H 20/10

(57) ABSTRACT

A portable medical data hub comprises a battery, at least one network interface, and a processor. The portable medical data hub is configured to: receive medical data from one or more medical devices via the at least one network interface; perform initial processing on the medical data using the processor to provide processed medical data; and transmit the processed medical data via the at least one network interface to a data analysis system for medical data analysis.

### CROSS REFERENCE TO RELATED APPLICATIONS

The present application is the national stage entry of International Patent Application No. PCT/EP2018/077429, filed on Oct. 9, 2018, and claims priority to Application No. EP 17306363.7, filed on Oct. 10, 2017, the disclosures of which are incorporated herein by reference.

### TECHNICAL FIELD

The application relates to a portable medical data hub, in particular, although not exclusively, to a portable medical data hub for acting as an interface between medical devices and the cloud.

### BACKGROUND

The measurement of medical data relating to a user's treatment by medical devices has become increasingly common. Such data may allow a third party to monitor the correct administration of medication or use of a device by a user, which is important for the safety and efficacy of the medication.

### SUMMARY

A portable medical data hub is provided, comprising: a battery; at least one network interface; a processor; wherein the intelligent medical interface device is configured to: receive medical data from one or more medical devices via the at least one network interface; perform initial processing on the medical data using the processor to provide processed medical data; and transmit the processed medical data via the at least one network interface to a data analysis system for medical data analysis.

A portable medical data hub thus provided may allow a user better to manage their treatment. It may also facilitate monitoring of the user's treatment by parties such as medical practitioners, whom may obtain information from the data analysis system. The portable medical data hub may allow a user better to treat themselves when multiple medical devices are involved in the treatment since it can allow the user to get information from the multiple medical devices to the data analysis system without requiring the user being able to operate, or provide, multiple wide area communication channels.

The portable medical data hub may further be configured to receive a response from the data analysis system in dependence on the medical data analysis.

Optionally, the initial processing of the medical data comprises tagging the medical data with additional data relating to the medical device use.

The portable medical data hub may further be configured to: receive medical data comprising sensitive and non-sensitive user data; separate the sensitive data from the non-sensitive data; and transmit the non-sensitive data to the data analysis system. In this way, a user's medical data may be kept private.

The portable medical may further configured to analyse the sensitive data using the processor arrangement. The portable medical data hub may also be configured to transmit the results of the sensitive data analysis to the data analysis system. This may further increase protection of the user's sensitive data.

Optionally, the portable medical data hub comprises a memory and is configured to store the medical data in the memory if no network connection to the data analysis system can be established, and to transmit the stored data once a network connection is established. This can increase the reliability of data transfer.

The portable medical data hub may further be configured to: receive updates for one or more medical devices from the data analysis system; and apply the updates to the one or more medical devices. This can help ensure that medical device software is kept up to date.

The portable medical data hub may further comprise a touch screen display. This can provide a convenient means through which a user can interact with the hub.

The portable medical data hub may further comprise a speaker, and be configured to provide spoken reminders to a user relating to the use of a medical device and/or medication. This can improve a user's compliance with a medical treatment.

The portable medical data hub may further comprise an optical code reader. The portable medical data hub may further comprise an RFID tag reader. These can allow medication to be registered and/or tracked with the hub.

The portable medical data hub may further comprise an electrical connector for connecting a medical device to the portable medical data hub, and wherein the portable medical data hub is configured to provide electrical power to the connected medical device via the electrical connector. This allows the hub to act a portable battery for medical devices.

Also provided herein is a medical data processing system comprising: the portable medical data hub of any preceding aspect; and one or more medical devices configured to transmit user data to the portable medical data hub.

The system may further comprise a data analysis system connected via a network to the portable medical data hub, wherein the data analysis system is configured analyse user data transmitted from the portable medical data hub to the remote server across the network to create analysed data and store the analysed data.

The specification describes a portable medical data hub, also called an intelligent interface device, for managing home used electronic medical devices.

These and other aspects of the invention will be apparent from and elucidated with reference to the embodiments described hereinafter.

### BRIEF DESCRIPTION OF THE FIGURES

Embodiments will now be described, by way of example only, with reference to the accompanying drawings, in which:

FIG. 1 is an isometric view of an example of a portable medical data hub according to embodiments of the specification;

FIG. 2 is an isometric view of the reverse side of the portable medical data hub of FIG. 1;

FIG. 3 is an example of a schematic representation of the electronics system of the portable medical data hub of FIG. 1;

FIG. 4 is a schematic representation of an example of a system for managing and processing medical data including the portable medical data hub of FIG. 1; and

FIG. 5 is a flowchart illustrating a method of using the portable medical data hub of FIG. 1; and

FIG. 6 illustrates a flowchart of a method of forwarding medical data received from a portable medical device by a portable medical data hub for analysis.

### DETAILED DESCRIPTION

Embodiments provide an apparatus, system and method for monitoring and analysing data collected by medical devices, particularly those relating to the administration of medication.

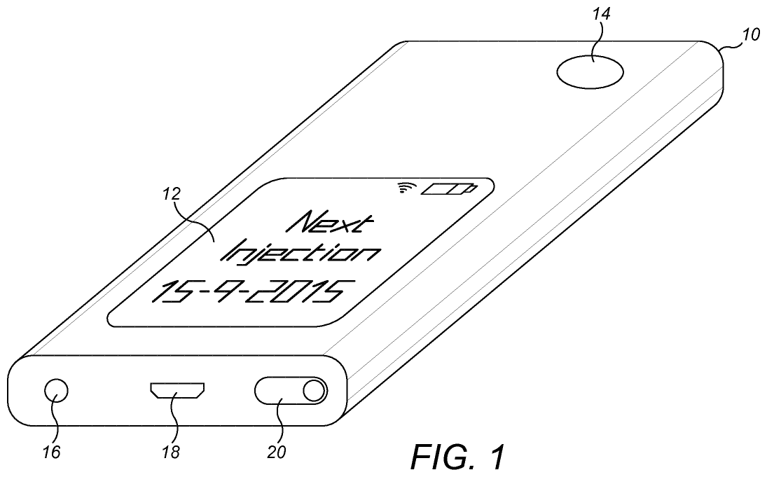

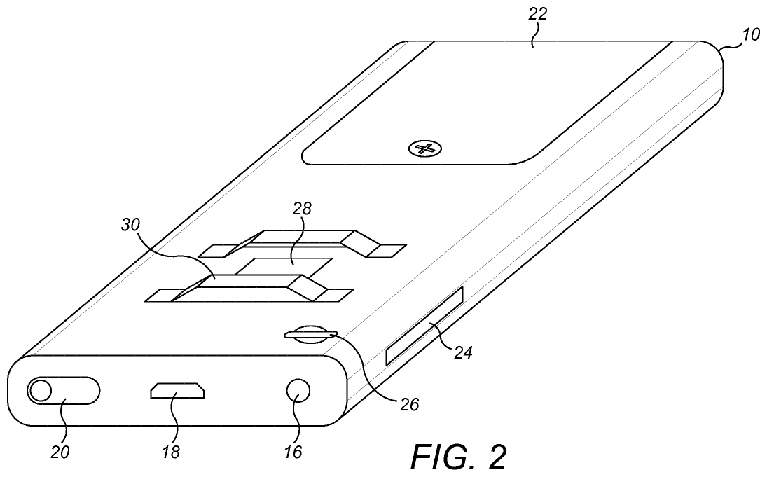

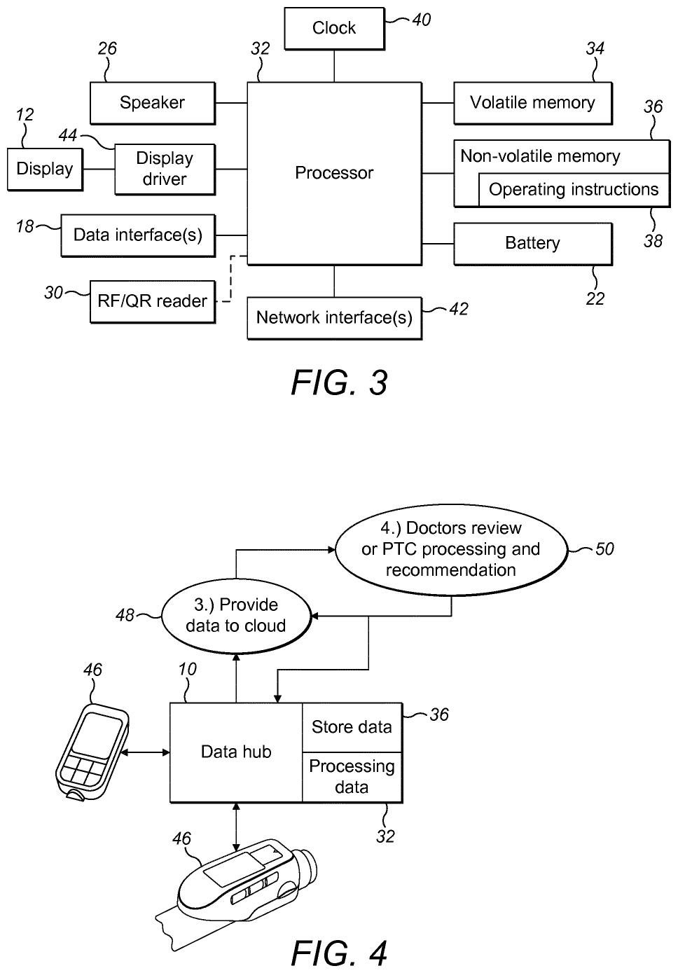

With respect to FIG. 1, an example of an intelligent medical device manager according to embodiments of the specification is shown in an isometric view. With respect to FIG. 2, an example of the reverse side of the FIG. 1 intelligent medical device manager is shown. With respect to FIG. 3, a schematic representation of the electronics system of the intelligent medical device manager of FIGS. 1 and 2 is shown.

Electronic medical devices are increasingly being used to monitor users' treatments. These devices can collect data relating the user and the user's treatment. However, often these devices have a limited computing power, so cannot fully utilise the data that they collect. Transmitting the collected data from the medical device to a portable medical data hub 10 (which may also be referred to as an intelligent medical device manager) allows the data to be analysed remotely from the medical device. The portable medical data hub 10 serves as a communication interface between medical devices and one or more external data processing systems, for example the cloud or a remote data server. The portable medical data hub 10 may also process the collected data itself.

The portable medical data hub 10 may be provided in a convenient portable size.

The portable medical data hub 10 comprises one or more network interfaces 42 for communicating with medical devices. The network interfaces 42 allow the portable medical data hub 10 to receive medical data from one or more medical devices. This may be, for example, via a network at the user location. For example, the data may be transmitted from the medical device to the user across a Wireless Local Area Network (WLAN) at the user location, or across a Bluetooth connection between the medical device and the portable medical data hub. The network interface 42 may also allow the portable medical data hub 10 to connect with other networks.

The portable medical data hub 10 further comprises a memory 36 for storing medical data received from medical devices. For example, if the portable medical data hub 10 receives medical data from an injection device relating to an administered injection, the portable medical data hub 10 may store the date and time of the injection and/or the dose details in the memory 36. In some embodiments, the memory 36 may contain a calendar relating to a user medication administration schedule. In some embodiments, the calendar and any injection record information stored in the device since the last device connection is synchronized automatically with a cloud database. This can reduce the amount of data stored on the portable medical data hub 10.

The portable medical data hub 10 also comprises a processing arrangement 32 for processing and/or analysing received medical data.

In some embodiments, the portable medical data hub 10 comprises a display 12. The display 12 can be operated by the processing arrangement 32 via a display driver 44 to provide one or more visual notifications to a user. The display 12 may in the form of an LCD screen. The display 12 may alternatively be in the form of an LED screen. The display 12 provides status information to the user relating to the portable medical data hub 10. Examples of such status information include which mode the apparatus is in, a battery status, a memory status, a network connection status and/or whether a power supply is connected. The display 12 may provide reminders to the user relating to the user's medication schedule. For example, a message may be provided on the display 12 stating when a user medication dose is scheduled to be administered. In some embodiments, the display 12 may provide messages relating to data transfers. These messages may comprise, for example, a message indicating that a successful or unsuccessful data transfer has occurred.

The display 12 may, in some embodiments, be a touch screen display. The user can interact with the portable medical data hub 10 through the touch screen display. Such interactions may comprise initiating a data transfer between a medical device and the portable medical data hub 10. The interactions may comprise initiating a data transfer between the portable medical data hub 10 and a data analysis system. The interaction may comprise dismissing a message provided to the user through the display 12.

The portable medical data hub 10 may comprise a speaker 26. The speaker 26 is an example of an audio transducer. The speaker 26 can be operated to provide an audio output in the form of spoken word, or more generally any sound. The speaker 26 may provide audible feedback to the user relating to the use of the portable medical data hub 10 and/or medical devices. An example of this would be an audible indication that a medical device was running low on medication contained in the device. In some embodiments, the speaker 26 may provide audible reminders to the user. For example, the speaker 26 may remind the user to take a scheduled dose of medication.

In some embodiments, one or more keys 14 may be provided on the portable medical data hub 10. The keys 14 may be used by a user to interact with the portable medical data hub 10. The keys may be provided as an alternative to or additionally to a touch screen display 12. One or more of the keys 14 may comprise a light, or more generally a visual indicator. The light 12 may be in the form of a ring around the key 14. The light may flash to indicate a status of the portable medical data hub 10. This may provide visual reminder functions, which can be useful for hearing impaired users. In some embodiments, the keys can be provided in the form of one or more buttons. The buttons can be provided with a visual feedback function.

The portable medical data hub 10 may comprise a jack socket 16, or more general an electrical connector. The jack socket 16 allows for charging of an external medical device using the portable medical data hub 10 battery. The portable medical data hub 10 can therefore act as a portable battery and/or charging system for external medical devices.

The portable medical data hub 10 may comprise a wired (non-wireless) data interface 18. The wired data interface 18 provides a means for wired connectivity with external devices. The external devices may include, for example, a medical device or a personal computer, such as a laptop computer, desktop computer, tablet computer etc. In some embodiments, a battery in the portable medical data hub 10 can be charged via the data interface 18. The wired data interface 18 may be in the form of a Universal Serial Bus (USB) port, for instance a micro-USB or USB-C port.

In some embodiments, an on/off switch 20 may be provided. This may be combined with keys 14 on the portable medical data hub such that, for example, holding a key 14 down for a period of time switches the portable medical data hub on or off, or alternatively be a stand-alone on/off switch.

The portable medical data hub 10 comprises a battery 22 for supplying power to the electronic systems of the portable medical data hub 10. The battery may be rechargeable, e.g. through the wired interface 18 or the jack socket 16, or it may be a replaceable battery. The battery 22 enables the use of the apparatus without a power connection to a mains supply.

The processor arrangement 32 may check the state of charge of one or more batteries 22. If the state of charge is determined to be low, the display 12 may be operated to indicate a battery low warning.

In some embodiments, an external memory interface 24 is provided on the portable medical data hub 10. The external memory interface 24 may allow for additional offline data storage using a removable memory. It may be provided, for example, in the form of an SD-Card slot, or another interface for connecting to a small form factor non-volatile memory device that is absent of a power supply. The external memory may serve to buffer medical data received by the portable medical data hub 10 when the portable medical data hub 10 is not connected to a network, and therefore unable to immediately transmit the data to a remote data analysis system.

The portable medical data hub 10 may, in some embodiments, comprise an RFID reader 28, or more generally a near field communications transceiver. RFID tags may then be read by the portable medical data hub 10. The reader 28 may alternatively or additionally include a QR-code reader, bar-code reader, or other optical code reader. The reader 28 may allow for the registration of medications that the user is using. For example, cartridges, auto-injectors (AIs), PFS and/or pills may be supplied with an RFID tag and/or optical code that can be read by the reader 28. This can provide information relating to the user's stock of medication, such as the amount of medication the user has been provided with or batch numbers of the medication.

The RFID reader comprises a radio frequency transceiver. The transceiver may be used to transmit power and data to a RFID tag in a medical device and to receive data from the RFID tag in response. In some embodiments, the RFID reader may be in the form of a NFC reader. Upon receipt of data from the RFID tag/QR code, the RFID/QR reader 28 may pass the received data to the processing arrangement 32. The processing arrangement 32 can process the received data and store it in the non-volatile memory 36 of the system for later retrieval. In some embodiments, the processing performed by the processing arrangement 32 comprises tagging the measured data with additional data before storage. For example, the measurement data may be tagged with the date and time of the measurement and/or the identity of the medical device from which the measurement was taken.

RFID orientation features 30 may be present in some embodiments. RFID orientation features 30 allow for correctly aligning an RFID tag in a medical device with an RFID reader 28 on the portable medical data hub 10. This may allow for the RFID tag to be read by the RFID reader 28 correctly.

It will be recognised by the skilled person that features provided on the reverse side of the portable medical data hub 10 may alternatively be provided on the front of the portable medical data hub 10, and vice versa.

As best seen from FIG. 3, the electronics system of the portable medical data hub comprises the processor arrangement 32. The processor arrangement 32 and other hardware components may be connected via a system bus (not shown). Each hardware component may be connected to the system bus either directly or via an interface. A power supply is arranged to provide power to the electronics system.

The processor arrangement 32 controls operation of the other hardware components of the electronics system. The processor arrangement 32 may be an integrated circuit of any kind. The processor arrangement 32 may for instance be a general purpose processor. It may be a single core device or a multiple core device. The processor arrangement 32 may be a central processing unit (CPU) or a general processing unit (GPU). Alternatively, it may be a more specialist unit, for instance a RISC processor or programmable hardware with embedded firmware. Multiple processors may be included. The processor arrangement 32 may be termed processing means.

The electronics system comprises a working or volatile memory 34. The processor arrangement 32 may access the volatile memory 34 in order to process data and may control the storage of data in memory. The volatile memory 34 may be a RAM of any type, for example Static RAM (SRAM), Dynamic RAM (DRAM), or it may be Flash memory. Multiple volatile memories may be included, but are omitted from the Figure.

The electronics system comprises a non-volatile memory 36. The non-volatile memory 36 stores a set of operation instructions for controlling the normal operation of the processor arrangement. The non-volatile memory 36 may be a memory of any kind such as a Read Only Memory (ROM), a Flash memory or a magnetic drive memory. Other non-volatile memories may be included, but are omitted from the Figure.

The processor arrangement 32 operates under the control of the operating instructions 38. The operating instructions 38 may comprise code (i.e. drivers) relating to the hardware components of the electronics system, as well as code relating to the basic operation of the apparatus. The operating instructions 38 may also cause activation of one or more software modules stored in the non-volatile memory 36. Generally speaking, the processor arrangement 32 executes one or more instructions of the operating instructions 38, which are stored permanently or semi-permanently in the non-volatile memory 36, using the volatile memory 34 temporarily to store data generated during execution of the operating instructions.

The processor arrangement 32, the volatile memory 34 and the non-volatile memory 36 may be provided as separate integrated circuit chips connected by an off-chip bus, or they may be provided on a single integrated circuit chip. The processor arrangement 32, the volatile memory 34 and the non-volatile memory 36 may be provided as a microcontroller.

The electronics system comprises a clock 40. The clock 40 may be a clock crystal, for example, a quartz crystal oscillator. The clock 40 may be a separate component to the processor arrangement 32 which is configured to provide a clock signal to the processor arrangement 32. The processor arrangement 32 may be configured to provide a real time clock based on the signal from the clock 40. Alternatively, the clock 40 may be a clock crystal which is provide on a single integrated circuit chip with the processor arrangement 32.

The electronics system comprises one or more network interfaces 42. The network interfaces 42 facilitate the connection of the apparatus to one or more computer networks and the bi-directional exchange of information between the apparatus and other members of the networks. These networks may include the Internet, a Local Area Network, or any other network required by the apparatus to communicate with the data centre and/or contact centre. The network interfaces 42 comprise a network interface controller, such as an Ethernet adaptor, a Wi-Fi adaptor and/or a Bluetooth adaptor. The network interfaces 42 are associated with one or more network addresses for identifying the apparatus on the network. The one or more network addresses may be in the form of an IP address, a MAC address, and/or an IPX address. Other members of the network may include medical devices that are collecting user data. The other members of the network may, in some embodiments, be connected to the portable medical data hub through Wi-Fi Protected Setup (WPS).

In some embodiments, the processor arrangement in the apparatus may not be sufficiently powerful to perform one or more of the functions described above. Instead, the processing arrangement is configured to communicate via the network interface with an additional computer system that has more computing power available to it. The processor arrangement can transmit data from the apparatus to the additional computer system, where it can be processed using the additional computing power of the additional computer system. The additional computer system can return the results of this processing back to the processor arrangement for further processing. The additional computing system can, for example, be a remote computer system, a distributed computer system, or part of a data centre.

FIG. 4 shows an example of a system for managing and processing medical data.

The system comprises one or more medical devices 46 comprising sensors for collecting user data, a portable medical data hub 10 as described above in relation to FIGS. 1 to 3, and a data analysis system 48 for processing/analysing the collected user data. The portable medical data hub 10 and data analysis system 48 are connected across a network in order to facilitate transfer of data between them.

In some embodiments, the data analysis system and/or portable medical data hub may additionally be connected to a third party computer system 50. The third party computer system 50 may be a second data analysis system. The third party computer system 50 may be associated with a healthcare professional who may be able to access the data analysis system 48 to review the processed data.

The system comprises one or more medical devices 46. Examples of such medical devices include an intelligent auto-injector (i-AI), a supplemental device for monitoring an injector device, a blood glucose meter (BGM) device, patch pumps and/or i-Pillboxes. The medical devices 46 may be equipped with sensors that collect medical data relating to the use of the medical device. The sensors may be integrated with the medical device. Alternatively or additionally, one or more of the medical device sensors may be provided on the medical devices in the form of detachable sensor.

When a user uses a medical device 46, the medical device sensors can measure data relating to that use. For example, the data may comprise the date and time of the medical device use, a dose of medication administered during the medical device use, and/or any user readings taken by the device. The data may comprise measurements taken on the user by the medical device. For example, the data may include blood pressure readings, pulse rates and/or blood sugar levels. A medical device 46 may comprise a memory to store the collected data until it can be transmitted to the portable medical data hub 10.

When a connection to the portable medical data hub 10 is established, the medical device 46 may transmit the recorded data to the portable medical data hub 10. The connection may, for example, be established over a WLAN at the user location or by a Bluetooth connection between the medical device 46 and the portable medical data hub.

In some embodiments, medical devices 46 may comprise RFID tags. The RFID tags can be read by an RFID reader on the portable medical data hub 10 to transfer medical data from the medical device 46 to the portable medical data hub 10.

Data transfer between a medical device 46 and the portable medical data hub 10 can, in some embodiments, be initiated by a user interaction with the medical device 46 or sensor on the medical device 46. For example, a key on the medical device 46 can be pressed to initiate the data transfer. In some embodiments, the data transfer can be initiated by the portable medical data hub. For example, the user may issue a command through the user interface on the portable medical data hub 10 (such as a touch screen display 12) to collect any medical data present in medical devices 46 that have a network connection with the portable medical data hub 10.

In some embodiments, the data transfer can be initiated automatically whenever a network connection between the medical device 46 and portable medical data hub 10 is detected, and collected data is determined be present in the medical device 46.

The data transferred from the medical device 46 to the portable medical data hub 10 can be processed by the processing arrangement 32 of the portable medical data hub 10. Such processing may involve tagging the data with additional data relating to the medical device use. Examples of said additional data may include the date and/or time of the medical device use, the identity of the medical device that the data was collected from, the identity of the measurement to which the data relates, and/or the identity of the user to which the data relates.

In some embodiments, the data transferred to the portable medical data hub 10 may include sensitive personal data relating to the user. The portable medical data hub 10 data processing may then comprise encryption of this data prior to transmission to the data analysis system 48. The portable medical data hub 10 data processing may comprise analysis of this sensitive data that would otherwise be performed at the data analysis system 48 instead of transmitting the sensitive data to the data analysis system 48. These features may assist in protecting the user's sensitive personal data.

The data transferred to the portable medical data hub 10 may be stored in the portable medical data hub memory 36 for later retrieval. Any additional data that the transferred data has been tagged with may also be stored.

When a network connection is established between the portable medical data hub 10 and the data analysis system 48, the portable medical data hub 10 can transmit the data to the data analysis system 48. The data may be transmitted directly from the portable medical data hub 10 to the data analysis system 48 upon receipt of the data from the medical device 46 if a network connection between the portable medical data hub 10 and the data analysis system 48 is present at that point. Previously collected data may be retrieved from the portable medical data hub memory 36 and transmitted to the data analysis system. The data transfer may occur automatically upon a network connection between the portable medical data hub 10 and the data analysis system 48 being established. The data transfer may alternatively or additionally be initiated upon some input from the portable medical data hub user. For example, the user may interact with the portable medical data hub 10 through a touch screen display on the portable medical data hub 10 to instruct the portable medical data hub 10 to transfer data to the data analysis system 48. In some embodiments, the data analysis system 48 may initiate the data transfer. The data analysis system 48 may, for example, periodically issue a request to the portable medical data hub 10 to transfer any data stored on the portable medical data hub 10 to the data analysis system.

The data analysis system 48 analyses the collected data. The results of such analysis may provide valuable data relating to a user medical activity. For example, by measuring the amount of a medication in a drug cartridge over time, a dose history for that medication can be created. This can also help track the compliance of a user with a medication regime. Taking measurements after each use of the drug cartridge may therefore be beneficial for monitoring the use of the drug by a user. Other examples include monitoring properties of the user over time to create records of the user's health. For example, variations in the user's weight, blood glucose and/or blood pressure over time may be recorded. This may better inform health care providers of the user's state of health.

The data analysis system 48 may comprise a user account for the portable medical data hub 10 user. The account comprises data collected by medical devices 42 relating to that user. It may comprise other information, such as data relating to the user identity. The collected data may be stored in the account in its raw and/or analysed forms. The data in the user account may be encrypted.

The data analysis system 48 may be provided as a server remote from the user location. In some embodiments, the data analysis system 48 may be provided as a distributed computing system, for example in the cloud.

In some embodiments, the system may comprise one or more connections to one or more third party computing systems 50. The third party computing system 50 may be a second data analysis system. Requests may be generated by the data analysis system based on the collected data and transmitted to the second data analysis system. Examples of a second data analysis system include an automated prescription service. The data analysis system 48 can issue a request to the automated prescription service for a medication to the sent to the user. The request may be generated as a result of determining from data collected from the medical devices 46 that a user may be low on a medication.

The third party computer system 50 may be a computer of a healthcare professional, who may be able to access the data analysis system 48 to review the processed data. The healthcare professional may access the data, for example, through a personal computer connected to the internet. The healthcare professional may provide updates to the data, such as a recommendation for the user based on the processed data. For example, the healthcare professional may suggest alterations to the user's medication regime. These alterations can be transmitted to the data analysis system and stored in the user account. The healthcare professional may be able to define the correct medication dosing into the user account. The system may transfer the dosage to medical devices and monitor the actually administered dosages.

The system may be provided with a calendar function relating to a user. Through the calendar function a forecast of drug needs for a period of time may be performed. For example, a prediction of how much of a medication a user should take on a holiday with them may be performed by the portable medical data hub 10 or a data analysis system 48 connected to the portable medical data hub 10, and the user informed. The system may calculate the drug needs of a user and may automatically request a prescription from a doctor or an automated prescription service. In some embodiments, the system may request a pharmacy to send the medication to the user in the event of the calendar determining a predicted shortage of a medication during a planned absence of the user.

The system may issue recommendations for the next dose of a medication. The system may issue a recommended time for the next dose to be administered. A reminder function can keep the user informed when an injection is due.

The system may allow for updates to medical device software to be provided to the medical devices 46 and/or the sensors in the medical devices 46. Updates can be transmitted from the data analysis system 48 to the portable medical data hub 10. The portable medical data hub then forwards the updates to the relevant medical device, for example when the portable medical data hub next detects that that device has a network connection with the portable medical data hub 10.

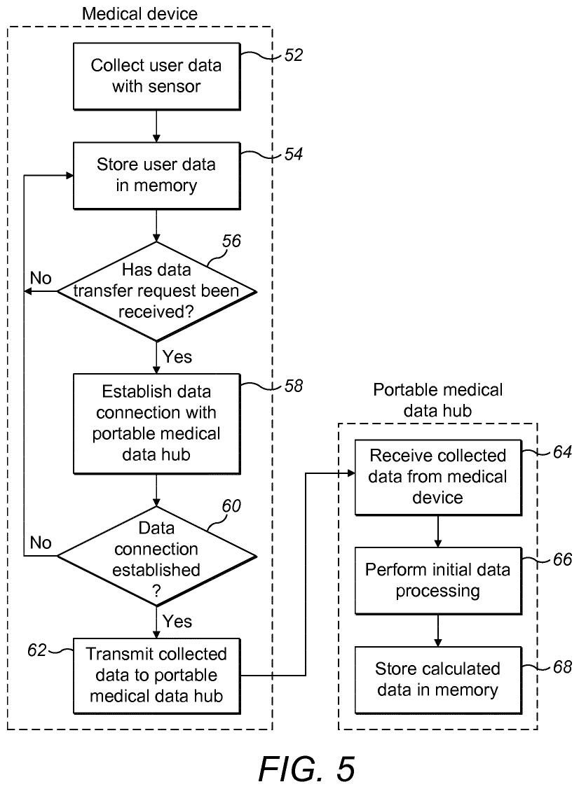

FIG. 5 is a flowchart illustrating a method of using the intelligent medical device manager of FIG. 1.

When a user uses a medical device 46 equipped with a sensor, the sensor collects data 52 relating to the use of the medical device. The medical device 46 then stores the data 54 in a memory of the medical device/sensor until it is transferred to the portable medical data hub.

When the medical device receives a request 56 to transfer the collected data to the portable medical data hub 10, it attempts to establish a data connection with the portable medical data hub 58.

The request 56 may, in some embodiments, be input directly into the medical device 46 through a user interface on the medical device. The request 56 may be generated automatically whenever the sensor on the medical collects data 52. A detection of a network connection between the medical device and a portable medical data hub may also act as the request 56 to transfer the collected data.

In some embodiments, the request 56 for data to be transferred from the medical device to the portable data hub may be received from the portable data hub. The request 56 may be generated in response to a user input to the portable medical data hub through a user interface (such as a touch screen). The portable medical data hub may also periodically issue requests 56 to medical devices with which it is connected.

In response to the request 56, the medical device attempts to establish a data connection 58 between the medical device and the portable medical data hub. If the data connection is determined to be established 60, then the collected data is retrieved from the medical device memory and transmitted 62 to the portable medical data hub 10.

If no data connection to a portable medical data hub is detected, then the medical device may store the data 56 in a memory of the medical device/sensor until a data connection is established.

Upon receipt of collected data 64, the portable medical data hub may perform initial processing of the data 66. The initial processing may comprises tagging the collected data with additional data 60. The collected data, along with any data resulting from the initial processing, is then stored in the portable medical data hub memory 68. The initial processing may comprise encrypting the collected data.

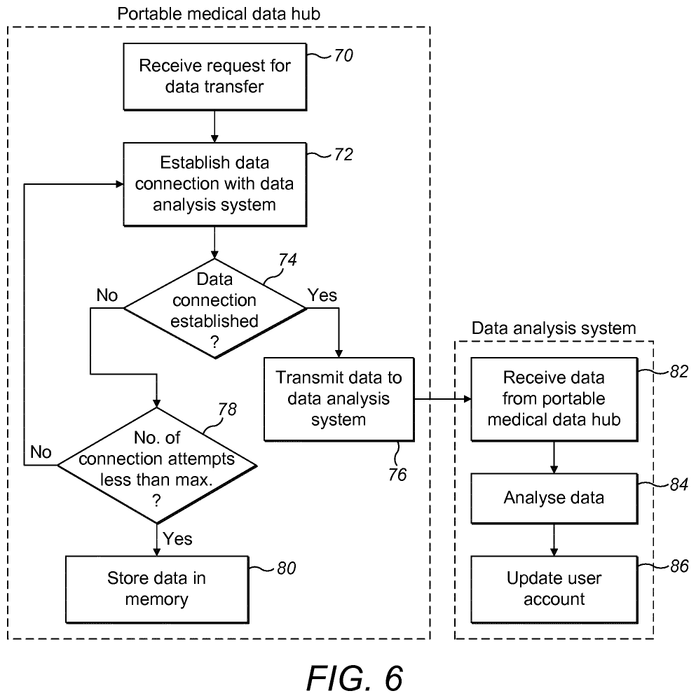

FIG. 6 illustrates a flowchart of a method of forwarding medical data from a portable medical device for analysis.

When the portable medical data hub 10 receives a request 70 for transfer of medical data stored in the memory of the portable medical data device to a data analysis system, the portable medical data hub attempts to establish a data connection 72 with the data analysis system.

The request 70 may be received by the portable medical data hub via a user interface on the portable medical data hub, such as a touch screen display. In some embodiments, it may be generated automatically on detection of a network connection between the portable medical data hub and the data analysis system.

The request 70 can, in some embodiments, be generated by the data analysis system and transmitted to the portable medical device. This may occur periodically, for example once an hour, once a day or once a week. Alternatively, it may occur when the data analysis system detects that a network connection is present between the portable medical data hub and the data analysis system.

The portable medical data hub then checks if a data connection between the portable medical data hub and the data analysis system has been established 74.

If a connection between the portable medical data hub and the data analysis system is determined to present, the data is retrieved from the portable medical data hub memory and transmitted to the data analysis system 76. The data may continue to be stored in the portable medical data hub memory. In some embodiments, the data may be deleted from the portable medical data hub memory after transmission. The data deletion may occur automatically upon completion of the data transfer to the data analysis system, or may require a user input before deletion. The user may be presented with an indication that the data transfer has been completed, for example via a display on the portable medical data hub. The indication may include a query as to whether the user would like the data to be deleted from the portable medical data hub memory. Upon a response from the user indicating that the data should be deleted, the portable medical data hub will delete the transferred data from the portable medical data hub memory.

If no data connection is established, the portable medical data hub may continue to attempt to establish a data connection to the data analysis system. The number of data connection attempts can be limited to prevent excessive use of the portable medical data hub battery power 78. If the number of connection attempts is below some predetermined value, then the portable medical data hub continues to try to establish a data connection to the data analysis system. If the number of connection attempts is above some predetermined value, then the portable medical data hub continues to store the requested data in the portable medical data hub memory 80.

In some embodiments, the collected data may comprise sensitive data that is not suitable for transmission across the network. In these embodiments, the sensitive data may be analysed by the portable medical data hub. The collected data may also comprise non-sensitive data. This may be separated from the sensitive data during the initial data processing 60. The non-sensitive data may then be transmitted to the data analysis system as described above. The results of the analysis of the sensitive data may be stored in the portable medical data hub memory for access by the user. The results of the analysis may be suitable for transmission across the network to the data analysis system. If so, the analysis results may be transmitted to the data analysis system for further analysis.

The transmitted data is received by the data analysis system 82.The data analysis system then analyses the transferred data 84 as described above in relation to FIG. 4. The user account in the data analysis system relating to the medical device user is then updated with the analysed and/or transferred data 86.

A response may be sent from the data analysis system to the portable medical data hub to indicate that the data has been successfully analysed. The response may provide the results of the analysis to the portable medical data hub. The results may be provided directly to the portable medical data hub for storage in the portable medical data hub memory. The user may then retrieve the results from the memory for viewing at a subsequent time. The results may be provided to the portable medical data hub in the form of a link to the user account on the data analysis system.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

1-15. (canceled)

16. A portable medical data hub, comprising:
a battery;
at least one network interface; and
a processor;
wherein the portable medical data hub is configured:
receive medical data from one or more medical devices via the at least one network interface,
perform initial processing on the medical data using the processor to provide processed medical data, and
transmit the processed medical data via the at least one network interface to a data analysis system for medical data analysis.

17. The portable medical data hub of claim 16, wherein the portable medical data hub is further configured to receive a response from the data analysis system in dependence on the medical data analysis.

18. The portable medical data hub of claim 16, wherein the initial processing of the medical data comprises tagging the medical data with additional data relating to use of the one or more medical devices.

19. The portable medical data hub of claim 16, wherein the medical data comprises sensitive and non-sensitive user data, wherein the portable medical data hub is configured to:
separate the sensitive data from the non-sensitive data; and
transmit the non-sensitive data to the data analysis system.

20. The portable medical data hub of claim 19, wherein the portable medical data hub is further configured to analyze the sensitive data using the processor.

21. The portable medical data hub of claim 20, wherein the portable medical data hub is configured to transmit, to the data analysis system, results of the analysis of the sensitive data.

22. The portable medical data hub of claim 16, wherein the portable medical data hub comprises a memory and is configured to
store the medical data in the memory if a network connection to the data analysis system cannot be established, and
transmit the stored data once the network connection is established.

23. The portable medical data hub of claim 16, configured to:
receive updates for the one or more medical devices from the data analysis system; and
apply the updates to the one or more medical devices.

24. The portable medical data hub of claim 16, further comprising a touch screen display.

25. The portable medical data hub of claim 16, wherein the portable medical data hub further comprises a speaker, and is configured to provide spoken reminders to a user relating to use of a medical device and/or medication.

26. The portable medical data hub of claim 16, further comprising an optical code reader.

27. The portable medical data hub of claim 16, further comprising an RFID tag reader.

28. The portable medical data hub of claim 16, further comprising an electrical connector for connecting a medical device to the portable medical data hub, and wherein the portable medical data hub is configured to provide electrical power to the connected medical device via the electrical connector.

29. A medical data processing system comprising:
a portable medical data hub comprising
a battery,
at least one network interface, and
a processor,
wherein the portable medical data hub is configured to:
receive medical data from one or more medical devices via the at least one network interface,
perform initial processing on the medical data using the processor to provide processed medical data, and
transmit the processed medical data via the at least one network interface to a data analysis system for medical data analysis; and
one or more medical devices configured to transmit user data to the portable medical data hub.

30. The medical data processing system of claim 29, further comprising:
a data analysis system connected via a network to the portable medical data hub, wherein the data analysis system is configured analyze user data transmitted from the portable medical data hub to a remote server across the network to create analyzed data and store the analyzed data.

31. The medical data processing system of claim 29, wherein the portable medical data hub is further configured to receive a response from the data analysis system in dependence on the medical data analysis.

32. The medical data processing system of claim 29, wherein the initial processing of the medical data comprises tagging the medical data with additional data relating to use of the one or more medical devices.

33. The medical data processing system of claim 29, wherein the medical data comprises sensitive and non-sensitive user data, wherein the portable medical data hub is configured to:
separate the sensitive data from the non-sensitive data; and
transmit the non-sensitive data to the data analysis system.

34. The medical data processing system of claim 33, wherein the portable medical data hub is further configured to analyze the sensitive data using the processor.

35. A method comprising:
receive, by a portable medical data hub, medical data from one or more medical devices via at least one network interface of the portable medical data hub,
perform, by the portable medical data hub, initial processing on the medical data to provide processed medical data, and
transmit, by the portable medical data hub, the processed medical data via the at least one network interface to a data analysis system for medical data analysis.
